(19) United States
(12) Patent Application Publication
(10) Pub. No.: US 2025/0344739 A1
(43) Pub. Date: Nov. 13, 2025

(54) MIX OF NON-DIGESTIBLE OLIGOSACCHARIDES

(71) Applicant: N.V. Nutricia, Zoetermeer (NL)

(72) Inventors: Cornelus Johannes Petrus van Limpt, Utrecht (NL); Ingrid Brunhilde Renes, Utrecht (NL); Lauriane Emmanuelle Mélanie Schwebel, Utrecht (NL)

(73) Assignee: N.V. Nutricia, Zoetermeer (NL)

(21) Appl. No.: 19/278,123

(22) Filed: Jul. 23, 2025

Related U.S. Application Data
(62) Division of application No. PCT/EP2024/053094, filed on Feb. 7, 2024.

(30) Foreign Application Priority Data
Feb. 7, 2023 (EP) 23155283.7

(51) Int. Cl. A23L 33/00; A23L 29/00; A23L 29/30; A61K 31/702; A61P 1/00

(52) U.S. Cl. CPC A23L 33/40; A23L 29/065; A23L 29/30; A61K 31/702; A61P 1/00; A23V 2400/519

(57) ABSTRACT

The present invention concerns nutritional compositions comprising a mixture of non-digestible oligosaccharides consisting of a combination of 5 specific human milk oligosaccharides, beta-galacto-oligosaccharides and long chain fructo-oligosaccharides (lcFOS). The nutritional compositions are of benefit for improving intestinal microbiota.

### FIELD OF THE INVENTION

The invention is in the field of nutritional compositions for infants and young children comprising non-digestible oligosaccharides to improve the intestinal microbiota.

### BACKGROUND OF THE INVENTION

Human milk contains substantial amounts of non-digestible carbohydrates, known as human milk oligosaccharides (HMOs). Mature human milk contains 5 to 15 g/l of HMOs. It is presumed that more than 200 structurally distinct oligosaccharides are present. The building blocks of human milk oligosaccharides are the monosaccharides D-glucose (Glc), D-galactose (Gal), N-acetylglucosamine (GlcNAc), L-fucose (Fuc), and sialic acid (N-acetyl neuraminic acid (Neu5Ac). Lactose (Galβ1-4Glc) forms the reducing end and can be elongated with N-acetyllactosamine repeat units (Galβ1-3/4GlcNAc). Lactose or the polylactosamine backbone can be sialylated in α2-3 and/or α2-6 linkages and/or fucosylated in α1-2, α1-3, and/or α1-4 linkages. The structural complexity and abundance of these non-digestible oligosaccharides is unique for human milk as in milk of other mammalian species the level of non-digestible oligosaccharides is much lower. The role of HMOs in human milk is to improve the intestinal microbiota by stimulating bifidobacteria and other beneficial lactic acid producing bacteria and thereby inhibiting the growth of potentially pathogenic bacteria. HMOs furthermore inhibit binding of pathogenic micro-organisms to the infant's epithelial cell surface, and also direct beneficial effects of HMOs on the gastro-intestinal epithelium an immune system have been reported.

Breastfeeding is the preferred method of feeding infants. However, there are circumstances that make breastfeeding impossible or less desirable. In those cases, infant formulas are a good alternative. The composition of modern infant formulas is adapted in such a way that it meets many of the special nutritional requirements of the fast growing and developing infant.

In the past, infant formulas did not contain non-digestible oligosaccharides. Subsequently infant formulas were developed, containing prebiotic, non-digestible oligosaccharides to functionally mimic the role of the HMOs. One of the best studied mixtures of such prebiotics is a mixture of galacto-oligosaccharides (GOS) plus long chain fructo-oligosaccharides (lcFOS) in a weight ratio of 9:1. Upon administration of this specific prebiotic mixture to infants, bifidobacteria increase and pathogens decrease in the intestinal microbiota, rendering the microbiota more similar to the microbiota of human milk fed infants (Knol et al, Acta Paadiatrica, 2005; 94 (Suppl 449); Knol et al, 31-33. Pediatr Gastroenterol Nutr, Vol. 40, No. 1, January 2005; WO 2005/039319).

More recently milk oligosaccharides with a structure identical to HMOs have become available as produced by fermentation by genetically modified micro-organisms and infant formulas containing a HMO or a mixture of HMOs have become available.

WO 2019/110800 discloses spray-dried mixtures of human milk oligosaccharide and nutritional compositions comprising this. Prebiotics such as galacto-oligosaccharides (GOS), fructo-oligosaccharides (FOS), inulin or combinations thereof may be included in the nutritional composition.

WO 2021/116236 discloses age staged nutritional compositions comprising a HMOs mix. Optionally, at least one of the nutritional compositions comprises a prebiotic, preferably the prebiotic comprises polydextrose, galacto-oligosaccharides, or a combination thereof.

WO 2020/239996 discloses the combination of galacto-oligosaccharides (GOS) and long chain fructo-oligosaccharides (lcFOS) and 2′-fucosyllactose (2′-FL) reduces or prevents proteolytic fermentation in the gastro-intestinal tract of a subject. The document shows that adding 2′-FL to the combination of GOS and lcFOS did not increase bifidobacteria achieved with the combination GOS/lcFOS alone.

WO 2020/245313 discloses that a combination of 2′-fucosyllactose (2′-FL) and 3′-galactosyllactose has a beneficial effect on the intestinal barrier function

WO 2019/055718 discloses the use of compositions to increase output of particular metabolites in the gut of a nursing infant mammal including humans. These compositions generally comprise one or more bacterial strains selected for their growth on mammalian milk oligosaccharides, a source of mammalian milk oligosaccharides, and, optionally, nutritive components required for the growth of that infant mammal.

CN 113907144 discloses that certain HMOs can prevent infection by Staphylococcus aureus. A wide array of HMO combinations is suggested including the combination of 2′-fucosyllactose (2′-FL), 3-fucosyllactose (3-FL), lacto-N-tetraose (LNT), 3′-sialyllactose (3′-SL) and 6′-sialyllactose (6′-SL). The document also discloses that in infant formula, dietary fiber such as fructooligosaccharides, galactooligosaccharides, polyfructsaccharides etc. may be included.

US 2014/248415 discloses HMO combinations comprising 2′-FL, LNT, 3′-SL and 6′-SL to be included in infant formula and discloses to optionally include as prebiotic 90% GOS, 10% inulin or FOS. The HMO combination is said to enhance the beneficial effects and efficiency of probiotics.

US 2020/354760 concerns a process for the purification of L-fucose from a fermentation to provide L-fucose in food-grade quality. In general terms a premix for an infant or toddler food formula is described that besides L-fucose comprises carbohydrate selected from the group consisting of a human milk oligosaccharide (HMO), a galactooligosaccharide (GOS), inulin, a fructooligosaccharide (FOS), lactose, isomaltose, sialic acid, and combinations thereof. As an example an infant formula is described that besides L-fucose comprises HMOs comprising 2′-FL, 3-FL, LNT, 3′-SL, 6′-SL.

US 2023/013644 concerns nutritional compositions comprising HMOs for use in providing nutrition to infants at different age stages. Infant formula with the combination of 2′-FL, 3-FL, LNT, 3′-SL, 6′-SL are described. The document suggests to further include prebiotics and examples of formula the comprise GOS and polydextrose (PDX) are given.

Parschat et al. Nutrients 2021 132871 reports on a clinical study assessing safety tolerability and effect on growth of infant formula containing a combination of 5 HMOs at 5.75 g/L comprising 52% 2′-FL, 13% 3-FL, 26/o LNT, 4% 3′-SL and 5% 6′-SL. The tested formula did not contain further prebiotics. The outcome was that the infant formula containing the combination of 5 HMOs supported normal infant growth and was safe and well tolerated.

Conze et al. Food and Chemical Toxicology 2022, 163:112377 made a weighted analysis of measurements of HMOs in human milk reported in literature and calculated the weighted means to be 54.2% 2′-FL, 12.0% 3-FL, 19.7% LNT, 5.9% 3′-SL and 8.2% 6′-SL. The authors take into consideration that HMOs are believed to play a pivotal role in balancing the intestinal microbiota, imparting antimicrobial effects, developing the intestinal barrier, and modulating immune response and in view of that the levels of the HMOs were found to vary widely, they suggest appropriate supplementation above mean levels found in human milk.

Still there is a need for infant and young child formula with a mixture of non-digestible oligosaccharides that further improves the intestinal microbiota of infants.

### SUMMARY OF THE INVENTION

Employing an in vitro fermentation model using infant faecal samples, the inventors surprisingly found that a mixture of non-digestible oligosaccharides consisting of a combination of 5 specific human milk oligosaccharides (5 HMOs), beta-galacto-oligosaccharides (bGOS) and long chain fructo-oligosaccharides (lcFOS), synergistically increased the amount of bifidobacteria and synergistically decreased the Gram-negative Enterobacteriaceae when compared to the mix of bGOS and lcFOS, or when compared to the combination of 5 HMOs alone. The combination of 5 specific HMOs is 2′-fucosyllactose (2′-FL), 3-fucosyllactose (3-FL), lacto-N-tetraose (LNT), 3′-sialyllactose (3′-SL) and 6′-sialyllactose (6′-SL). The synergistic effects were consistently found when faecal samples of several infants were tested. Furthermore, when testing the fermentation supernatants in a model for the intestinal barrier, the barrier resistance was found to be synergistically increased in case of the fermentation supernatant of the present mixture of non-digestible oligosaccharides. Therefore, a nutritional composition comprising a mixture of 5 specific HMOs, bGOS and lcFOS will have a further improved effect on the intestinal microbiota by increasing bifidobacteria or decreasing pathogenic bacteria and an improved effect on the intestinal barrier function. The risk of gastrointestinal disorders such as microbial dysbiosis, infections and inflammation will be further reduced.

### DETAILED DESCRIPTION OF THE INVENTION

The present invention concerns a nutritional composition for infants or young children comprising a mixture of non-digestible oligosaccharides consisting of
component a): beta-galacto-oligosaccharides (bGOS) and long chain fructo-oligosaccharides (lcFOS), and
component b): 2′-fucosyllactose (2′-FL), 3-fucosyllactose (3-FL), lacto-N-tetraose (LNT), 3′-sialyllactose (3′-SL) and 6′-sialyllactose (6′-SL). Component b) can also be referred to as combination of 5 HMOs.

The invention also concerns the present nutritional composition for use in therapy.

The invention also concerns the use of the present nutritional composition for improving the intestinal microbiota, preferably in infants or young children.

The invention also concerns the use of the present nutritional composition for increasing the intestinal barrier function, preferably in infants or young children.

The invention also concerns the use of the present nutritional composition for preventing an/or treating an intestinal disorder.

### Component a): Beta-Galacto-Oligosaccharides and Long Chain Fructo-Oligosaccharides

The nutritional composition comprises the non-digestible oligosaccharides beta-galacto-oligosaccharides (bGOS) and long chain fructo-oligosaccharides (lcFOS). Non-digestible oligosaccharides are oligosaccharides with an average degree of polymerization ranging from 2 to 100. Non-digestible oligosaccharides are oligosaccharides that are nondigested in the stomach or small intestine and reach the colon intact. Maltodextrin, lactose and monomers such as galactose, fucose, and sialic acid are not considered non-digestible oligosaccharides, i.e. they are considered digestible carbohydrates.

The present nutritional composition comprises beta-galacto-oligosaccharides (bGOS). bGOS are non-digestible oligosaccharides preferably having the formula ([galactose]n-glucose; wherein n is an integer ranging from 2 to 10, i.e. 2, 3, 4, 5, 6, . . . , 10), wherein the galactose units are in majority linked together via a beta linkage. bGOS are for example sold under the trademark Vivinal™ GOS (Borculo Domo Ingredients, Netherlands). Other suitable sources are Oligomate™ (Yakult, Japan). Preferably the present bGOS have an average degree of polymerization (DP) ranging from 3 to 7, more preferably ranging from 3 to 5. Preferably the bGOS comprise mainly beta-1,4 linkages and/or beta-1,6 linkages between the galactose units, more preferably predominantly beta-1,4 linkages. In a preferred embodiment, the bGOS comprise at least 80% beta-1,4 and beta-1,6 linkages based on total linkages, more preferably at least 90%. bGOS, are more capable of stimulating bifidobacteria. Preferably the bGOS comprises less than 10% beta 1,3 linkages based on total linkages.

The present nutritional composition comprises long chain fructo-oligosaccharides (lcFOS). lcFOS are non-digestible oligosaccharides comprising a chain of beta-linked fructose units with an average degree of polymerization (DP) ranging from 6 to 1000, more preferably 10 to 100, even more preferably 20 to 40. lcFOS include inulin, levan and/or a mixed type of polyfructan. An especially preferred lcFOS is inulin. Inulin has a structure of chain-terminating glucosyl moieties and a repetitive fructosyl moiety, which are linked by beta-2,1 linkages. lcFOS suitable for use in the present nutritional composition is also commercially available, e.g. Raftiline®HP (Orafti).

For the purpose of this invention, when amounts or ranges are expressed per volume this refers to the nutritional composition in a ready-to-use form, unless expressed otherwise. When amounts are expressed as wt %, this refers to the wt % based on dry weight, unless expressed otherwise.

Preferably, the present nutritional composition comprises 100 mg to 2 g bGOS plus lcFOS per 100 ml, more preferably 250 mg to 1.5 g, even more preferably 500 mg to 1 g per 100 ml. Based on dry weight, the present nutritional composition preferably comprises 0.7 wt % to 14.3 wt %, more preferably 1.8 to 10.7 wt %, even more preferably 3.6 to 7.1 wt % bGOS plus lcFOS. Based on 100 kcal the present nutritional composition preferably comprises 150 mg to 3 g bGOS plus lcFOS, more preferably 375 mg to 2.25 g, even more preferably 750 mg to 1.5 g. Preferably, the present nutritional composition comprises less than 10 mg beta1,3′-galactosyllactose (3′-GL) per 100 ml. Based on dry weight, the present nutritional composition preferably comprises less than 0.07 wt % 3′-GL. Based on 100 kcal the present nutritional composition preferably comprises less than 15 mg 3′-GL.

Based on total weight of bGOS plus lcFOS, preferably the amount of bGOS ranges from 70 to 95% and the amount of lcFOS ranges from 5 to 30%, the sum of bGOS and lcFOS being 100%. More preferably based on total weight of bGOS plus lcFOS, the amount of bGOS ranges from 85 to 95% and the amount of lcFOS ranges from 5 to 15%, the sum of bGOS and lcFOS being 100%. It is preferred to combine GOS with lcFOS instead of other longer oligosaccharides such as polydextrose. A mixture of bGOS and lcFOS was found to have a synergistic effect on the amount of intestinal bifidobacteria and lactobacilli and formation of short chain fatty acids when compared to bGOS or lcFOS alone. Also mixtures of GOS with lcFOS were shown to produce higher amounts of short chain fatty acids and lactic acid than wen polydextrose was used (Vester-Boler et al., Nutr Res 2009, 29:631-639).

### Component b): Combination of 5 HMOs

The nutritional composition according to the invention comprises a specific combination of human milk oligosaccharides consisting of 2′-fucosyllactose (2-′FL), 3-fucosyllactose (3-FL), lacto-N-tetraose (LNT), 3′-sialyllactose (3′-SL) and 6′-sialyllactose (6′-SL). It was found that this specific mix is superior in combination with component a). A combination of 4 HMOs instead of 5 HMOs showed less effect. As the fermentation of HMOs by intestinal microbiota results in interactions such as cross feeding and quorum sensing, the outcome of one specific combination of HMOs cannot be extrapolated to HMOS combinations that are different.

The above HMOs may be isolated by chromatography or filtration technology from a natural source such as animal milks. Alternatively, they may be produced by biotechnological means using specific enzymes such as fucosyltransferases and/or fucosidases for the production of 2′-FL and 3-FL, sialidases and glycosyltransferases for SLs and LNT, either through the use of enzyme-based fermentation technology (recombinant or natural enzymes) or microbial fermentation technology known in the art. In the latter case, microbes may either express their natural enzymes and substrates or may be engineered to produce respective substrates and enzymes. Single microbial cultures and/or mixed cultures may be used. Alternatively, these HMOs may be produced by chemical synthesis, for example from lactose and free monomers such as fucose, sialic acid, N acetyl glucosamine, galactose. These HMOs are commercially available, for example from Kyowa Hakko, Japan, FrieslandCampina, The Netherlands, Glycom/DSM, Denmark, Chr. Hansen, Denmark and Sigma-Aldrich.

The amount of the above combination of 5 HMOs is preferably 20 to 400 mg per 100 ml, more preferably 30 to 300 mg per 100 ml, even more preferably 40 to 250 mg per 100 ml. Based on dry weight of the composition the amount of human milk oligosaccharides is preferably 0.14 to 2.86 wt %, more preferably 0.21 to 2.14 wt %, even more preferably 0.28 to 1.79 wt %. Preferably the amount of human milk oligosaccharides per 100 kcal is 30 to 600 mg, more preferably 45 to 450 mg, even more preferably 60 to 375 mg.

The amount of each specific HMO is preferably, based on total weight of HMOs, 42 to 62 wt % 2′-FL, 10 to 16 wt % 3-FL, 21 to 31 wt % LNT, 3 to 5 wt % 3′-SL, 4 to 6 wt % 6′-SL, the sum of 2′-FL, 3-FL, LNT, 3′-SL and 6′-SL being 100%, more preferably 47 to 57 wt % 2′-FL, 11 to 15 wt % 3-FL, 23 to 28 wt % LNT, 3.5 to 4.5 wt % 3′-SL and 4.5 to 5.5 wt % 6′-SL. Such ratios will ensure an even better effect on the microbiota.

Mixture of Combination of HMOs and bGOS and lcFOS

The nutritional composition of the present invention comprises a mixture of non-digestible oligosaccharides consisting of bGOS, lcFOS, 2′-FL, 3-FL, LNT, 3′-SL and 6′-SL. It was found that such a mixture synergistically improves the microbiota and the intestinal barrier resistance. As this effect on improving the microbiota is complex and thought to be specific all 7 non-digestible oligosaccharides should be present and deletion or addition of further non-digestible oligosaccharides may yield different results.

Preferably the nutritional composition of the present invention comprises a mixture of non-digestible oligosaccharides consisting of
component a) beta-galacto-oligosaccharides (bGOS) having an average degree of polymerization (DP) ranging from 3 to 7, and long chain fructo-oligosaccharides (lcFOS) having an average degree of polymerization (DP) ranging from 20 to 40, and
component b) (combination of 5 HMOs) 2′-fucosyllactose (2′-FL), 3-fucosyllactose (3-FL), lacto-N-tetraose (LNT), 3′-sialyllactose (3′-SL) and 6′-sialyllactose (6′-SL),
wherein component a) consists of 70 to 95 wt % bGOS, based on total weight of component a), and 5 to 30 wt % lcFOS, based on total weight of component a), the sum of bGOS and lcFOS being 100% of the total weight of component a), and
wherein component b) consists of 42 to 62 wt % 2′-FL, 10 to 16 wt % 3-FL, 21 to 31 wt % LNT, 3 to 5 wt % 3′-SL and 4 to 6 wt % 6′-SL, the sum of 2′-FL, 3-FL, LNT, 3′-SL and 6′-SL being 100% of the total weight of component b).

The amount of non-digestible oligosaccharides is preferably 120 mg to 2.40 g per 100 ml, more preferably 280 mg to 1.80 g per 100 ml, even more preferably 540 mg to 1.25 g per 100 ml. Based on dry weight of the composition the amount non-digestible oligosaccharides is preferably 0.86 to 17.14 wt %, more preferably 2.00 to 12.86 wt %, even more preferably 3.86 to 8.93 wt %. Preferably the amount of non-digestible oligosaccharides per 100 kcal is 180 mg to 3.60 g, more preferably 420 mg to 2.70 g, even more preferably 810 mg to 1.88 g.

In a preferred embodiment, in the nutritional composition of the present invention, the amount of bGOS plus lcFOS is 100 mg to 2 g per 100 ml, more preferably 250 mg to 1.5 g, even more preferably 500 mg to 1 g per 100 ml; based on dry weight of the composition the amount of bGOS plus lcFOS is 0.7 wt % to 14.3 wt %, more preferably 1.8 to 10.7 wt %, even more preferably 3.6 to 7.1 wt %; and/or the amount of bGOS plus lcFOS per 100 kcal is 150 mg to 3 g, more preferably 375 mg to 2.25 g, even more preferably 750 mg to 1.5 g; and

the amount of the combination of 5 HMOs is 20 to 400 mg per 100 ml, more preferably 30 to 300 mg per 100 ml, even more preferably 40 to 250 mg per 100 ml; based on dry weight of the composition the amount of the combination of 5 HMOs is 0.14 to 2.86 wt %, more preferably 0.21 to 2.14 wt %, even more preferably 0.28 to 1.79 wt %; and/or the amount of the combination of 5 HMOs per 100 kcal is 30 to 600 mg, more preferably 45 to 450 mg, even more preferably 60 to 375 mg.

Preferably the nutritional composition according to the present invention comprises 50 to 97.5 wt % of component a) based on total non-digestible oligosaccharides and 2.5 to 50 wt % of component b) based on total non-digestible oligosaccharides, the sum of component a) and b) being 100% of the total non-digestible oligosaccharides. More preferably the present nutritional composition comprises 60 to 95 wt % of component a) based on total non-digestible oligosaccharides and 5 to 40 wt % of component b) based on total non-digestible oligosaccharides, the sum of component a) and b) being 100%. Most preferably the present nutritional composition comprises 75 to 90 wt % of component a) based on total non-digestible oligosaccharides and 10 to 25 wt % of component b) based on total non-digestible oligosaccharides, the sum of component a and b being 100%. Preferably the weight ratio of components a/b ranges from 39 to 1, more preferably ranges from 19 to 1.5, even more preferably ranges from 9 to 3. Such ratios will result in further improved effects on the microbiota and the intestinal barrier function.

Preferably, based on total weight, the mixture of non-digestible oligosaccharides consists of 60 to 90 wt % bGOS, 7.5 to 10 wt % lcFOS, 1.5 to 15 wt % 2′-FL, 0.4 to 4 wt % 3-FL, 0.75 to 7.5 wt % LNT, 0.10 to 1.2 wt % 3′-SL, and 0.10 to 1.5 wt % 6′-SL, the sum being 100%, more preferably, based on total weight, the mixture of non-digestible oligosaccharides consists of 65 to 85 wt % bGOS, 7.5 to 9.5 wt % lcFOS, 5 to 12 wt % 2′-FL, 1 to 3 wt % 3-FL, 2 to 6 wt % LNT, 0.2 to 1.0 wt % 3′-SL, and 0.20 to 1.2 wt % 6′-SL. Such ratios will result in further improved effects on the microbiota and the intestinal barrier function.

### Nutritional Composition

The nutritional composition according to the invention is not native cow's milk or native milk from another mammal. The present nutritional composition preferably comprises digestible carbohydrates, protein and lipid, wherein the lipid preferably provides 30 to 60%, preferably 35 to 55% of the total calories, the protein provides 5 to 15%, more preferably 6 to 12%, even more preferably 7 to 9% of the total calories and the digestible carbohydrates provide 25 to 75%, more preferably 40 to 60% of the total calories. Non-digestible oligosaccharides have a caloric density of 2 kcal/g and preferably make up 0.4 to 7% of total calories. The nutritional composition preferably comprises 3 g to 7 g lipid/100 kcal, preferably 4 g to 6 g lipid/100 kcal, more preferably 4.5 g to 5.5 g lipid/100 kcal; it preferably comprises 1.25 g to 4 g protein/100 kcal, more preferably 1.5 g to 3.0 g protein/100 kcal, more preferably 1.8 g to 2.2 g protein/100 kcal and it preferably comprises 6 g to 20 g digestible carbohydrate/100 kcal, more preferably 10 g to 15 g digestible carbohydrate/100 kcal.

Preferably the nutritional composition has an energy density of 45 to 75 kcal/100 ml, more preferably 60 to 70 kcal/100 ml, even more preferably 65 to 70 kcal/100 ml when in a ready-to-use form. This density ensures an optimal balance between hydration and caloric intake.

The nutritional composition is preferably a solid product, preferably a powder. Suitably, the nutritional composition is in a powdered form, which can be reconstituted with water, to form a ready-to-use liquid. Alternatively, the nutritional composition may be a ready-to-use liquid or is in a liquid concentrate form that should be diluted with water to a ready-to-use liquid.

The nutritional composition preferably comprises digestible carbohydrates. Based on calories the nutritional composition preferably comprises 6 g to 20 g digestible carbohydrates per 100 kcal, more preferably 10 g to 15 g per 100 kcal. When in liquid form, e.g. as a ready-to-use liquid, the nutritional composition preferably comprises 4 g to 15 g digestible carbohydrate per 100 ml, more preferably 7 g to 10 g per 100 ml. Based on dry weight the nutritional composition preferably comprises 30 wt % to 85 wt %, more preferably 40 wt % to 65 wt % digestible carbohydrates. Worded alternatively, when the nutritional composition is in powder form, the digestible carbohydrates are preferably present in an amount of 40 g to 85 g/100 g dry weight, more preferably 40 g to 65 g/100 g dry weight. Preferred digestible carbohydrate sources are one or more of lactose, glucose, sucrose, fructose, galactose, maltose, starch and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt %, more preferably at least 50 wt %, more preferably at least 75 wt %, even more preferably at least 90 wt %, most preferably at least 95 wt % of the digestible carbohydrate is lactose.

The nutritional composition preferably comprises protein. The protein concentration in the nutritional composition is determined by the sum of protein, peptides and free amino acids. Preferably the nutritional composition comprises 1.25 g to 4 g protein per 100 kcal, even more preferably 1.5 g to 3.0 g protein per 100 kcal, even more preferably 1.8 to 2.2 g per 100 kcal. A low protein concentration advantageously is closer to human milk as human milk comprises a lower amount of protein based on total calories compared to cow's milk. Based on a ready-to-use liquid product the nutritional composition preferably comprises 0.8 to 2.5 g per 100 ml, more preferably 1.0 g to 2.0 g/100 ml, even more preferably 1.2 to 1.5 g per 100 ml. Based on dry weight the nutritional composition preferably comprises 6 to 18 wt %, more preferably 7 to 15 wt %, even more preferably 8 to 11 wt % protein. Worded alternatively, when the nutritional composition is in powder form, the proteins are preferably present in an amount of 6 g to 18 g/100 g dry weight, more preferably 7 to 15 g, even more preferably 8 to 11 g/100 g dry weight of the composition. The source of the protein is preferably selected in such a way that the minimum requirements for essential amino acid content are met and satisfactory growth is ensured. Hence protein sources based on cows' milk proteins such as whey, casein and mixtures thereof and proteins based on soy, rice or pea are preferred. In case whey proteins are used, the protein source is preferably based on acid whey or sweet whey, modified sweet whey, whey protein isolate or mixtures thereof.

The nutritional composition of the present invention preferably comprises lipids. The lipid is preferably present in an amount of 3 g to 7 g per 100 kcal, more preferably in an amount of 4 g to 6 g lipid per 100 kcal and most preferably in an amount of 4.5 g to 5.5 g lipid per 100 kcal. When in liquid form, e.g. as a ready-to-use liquid, the nutritional composition preferably comprises 2.2 g to 4.5 g lipid per 100 ml, more preferably 2.5 to 4.0 g, even more preferably 3.0 to 3.75 g per 100 ml. Based on dry weight the nutritional composition preferably comprises 16 to 32 wt %, more preferably 18 to 30 wt %₆, even more preferably 20 to 28 wt % lipid. Worded alternatively, when the nutritional composition is in powder form, the lipids are preferably present in an amount of 16 g to 32 g/100 g dry weight, more preferably 18 to 30 g, even more preferably 20 to 28 g/100 g dry weight of the composition.

The lipid preferably comprises vegetable lipid. The presence of vegetable lipid advantageously enables an optimal fatty acid profile high in polyunsaturated fatty acids, such as essential linolenic acid and alpha-linolenic acid, and is more reminiscent to human milk fat. Lipid from non-human mammalian milk alone, e.g. cow milk, does not provide an optimal fatty acid profile. The amount of essential fatty acids is too low in non-human mammalian milk. Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, soy oil, palm oil and palm kernel oil.

Additionally animal fat such as cow's milk fat is preferably present in the nutritional composition. Such sources of lipid may provide additional desired components such as butyric (BA) and caproic acid (CA) and beta-palmitic acid (sn2-PA). Components such as butyric acid are known to have a synergistic effect together with human milk oligosaccharides on the gut barrier, immune system and anti-pathogenic effect. Preferably the nutritional composition comprises at least 0.5 wt % butyric acid based on total fatty acids, more preferably at least 0.7 wt % to 2 wt %.

Additionally egg oil and/or fish oil and/or microbial oils such as oil from fungi and algae may be present. Such oils are suitable sources of long-chain polyunsaturated fatty acids such as docosahexaenoic acid (DHA), arachidonic acid (ARA) and/or eicosapentaenoic acid (EPA). Preferably the nutritional composition comprises n3 LC-PUFA, such as EPA and/or DHA, more preferably DHA. Preferably the nutritional composition comprises at least 0.05 wt %, preferably at least 0.1 wt %, more preferably at least 0.2 wt %, DHA based on total fatty acids. Preferably the nutritional composition comprises not more than 2.0 wt %, preferably not more than 1.0 wt % DHA based on total fatty acids. The nutritional composition preferably comprises ARA. Preferably the nutritional composition comprises at least 0.05 wt %, preferably at least 0.1 wt %, more preferably at least 0.2 wt % ARA based on total fatty acids.

Preferably further ingredients are present in the nutritional composition like vitamins, minerals, trace elements, nucleotides and other micronutrients as known in the art.

Preferably the nutritional composition comprises lactic acid producing bacteria selected from the group consisting of the genera Bifidobacterium and/or Lactobacillus, in particular Bifidobacterium. The intestinal microbiota of breastfed infants is high in bifidobacteria. Addition to the nutritional composition of one or more strains belonging to the Bifidobacterium genus will further improve the intestinal microbiota and its activity. More preferably the nutritional composition comprises Bifidobacterium breve. Such strains of the species of Bifidobacterium breve are commercially available or can be isolated from the microbiota of infants. An example of a commercially available strain is Bifidobacterium breve M-16V from Morinaga. The amount of Bifidobacterium and/or Lactobacillus is preferably 10⁴ to 10¹¹ cfu per gram dry weigh of the nutritional composition.

The nutritional composition according to the invention is preferably in the form an infant formula, a follow-on formula or a young child formula. This means that the composition that is to be administered is not human milk. Infant formula or follow-on formula or young child formula means that it concerns a composition that is artificially made or in other words that it is a synthetic composition. In the context of the present invention, young child formula can also be named growing-up milk. The present nutritional composition preferably is intended for, or is used for, providing nutrition to an infant or young child,

Infant formulas are intended for infants from birth to about 4 to 6 months of age and are intended as a substitute for human milk. Typically, infant formulae are suitable to be used as sole source of nutrition. Such infant formulae are also known as starter formula. In the context of the present invention this is referred to as a nutritional composition, or infant formula, for the first 6 months of life.

Follow-on formulas are intended for infants staring with 4 to 6 months of age to 12 months of age and are intended to be supplementary feedings for infants that start weaning on other foods. In the context of the present invention this is referred to as a nutritional composition, or follow-on formula, for the age of 6 to 12 months.

Young child formula refers to nutritional compositions, artificially made, intended for children of 12 months to 36 months, in other words for children of 1 to 3 years of age which are intended to be supplementary feedings. In the context of the present invention this is referred to as a nutritional composition, or young child formula, for the age of 12 to 36 months, for the age of 1 to 3 years.

Infant formulae and follow-on formulae are subject to strict regulations, for example for the EU regulations no. 609/2013 and no. 2016/127 and Codex Alimentarius for Infant Formula, CODEX STAN 72-1981. Young child formulas preferably follow the directive for follow-on formula.

The nutritional composition is preferably an infant formula or a follow-on formula.

When the nutritional composition is in the form of an infant formula, the nutritional composition preferably comprises 0.25 g to 1.50 g per 100 ml, more preferably 0.50 to 1.00 g per 100 ml of component a) and preferably 0.03 to 0.40 g, more preferably 0.04 to 0.30 g per 100 ml of component b). The weight ratio a/b when the nutritional composition is in the form of an infant formula is preferably in the range of 2 to 9, more preferably in the range of 3 to 6.

When the nutritional composition is in the form of a follow-on formula, the nutritional composition preferably comprises 0.25 to 1.50 g per 100 ml, more preferably 0.50 to 1.00 g per 100 ml of component a) per 100 ml, and preferably 0.03 to 0.30 g, more preferably 0.04 to 0.25 g per 100 ml of component b). The weight ratio a/b when the nutritional composition is in the form of a follow-on formula is preferably in the range of 5 to 10, more preferably in the range of 7 to 9.

When the nutritional composition is in the form of a young child formula the nutritional composition preferably comprises 0.50 to 2.00 g, more preferably 0.50 to 1.50 g per 100 ml of component a), and preferably 0.02 to 0.30 g, more preferably 0.03 to 0.25 g per 100 ml of component b). The weight ratio a/b when the nutritional composition is in the form of a young child formula is preferably in the range of 15 to 39, more preferably in the range of 20 to 30.

Young children are in need of a higher amount of non-digestible oligosaccharides as the microbiota is more difficult to correct due to the formulas playing less of a role in the total daily diet than for example an infant formula that can be the sole form of nutrition. Also looking at human milk, the amount of HMOs reduces during lactation with increase of the age of the infant.

### Application

The nutritional composition of the present invention is preferably used for providing nutrition to an infant or young child, preferably an infant. An infant is a human child under the age of 12 months. A young child is a human child aged between one and three years, also called a toddler.

Preferably the nutritional composition of the present invention is provided to a human subject during the first 3 years of life. Preferably the nutritional composition is used in a method for providing nutrition to a human subject in the first 12 months of life, optionally the first 3 years of life, comprising providing a first infant formula for the first 6 months of life, wherein the formula comprises the mixture of non-digestible oligosaccharides of the present invention, wherein component a) provides 0.25 to 1.50 g per 100 ml, more preferably 0.50 to 1.00 g, and component b) provides 0.03 to 0.40 g, preferably 0.04 to 0.30 g per 100 ml, providing a second follow-on formula for the age of 6 to 12 months, wherein the formula comprises the mixture of non-digestible oligosaccharides of the invention, wherein component a) provides 0.25 to 1.2 g per 100 ml, more preferably 0.50 to 1.00 g, and component b) provides 0.03 to 0.30 g, preferably 0.04 to 0.25 g per 100 ml, and optionally providing a third young child formula, for the age of 12 to 36 months, i.e. 1 to 3 years, the third formula comprising the mixture of non-digestible oligosaccharides of the invention, wherein component a) provides 0.50 to 2.00 g per 100 ml, more preferably 0.50 to 1.50 g, and component b) provides 0.02 to 0.30 g, preferably 0.03 to 0.25 g per 100 ml, with the proviso that the weight ratio a/b of the second formula is higher than that of the first formula, and the weight ratio a/b of the optional third formula is higher than that of the second infant formula. Preferably the weight ratio a/b is from 2 to 9 for the first formula, from 5 to 10 for the second formula and from 15 to 39 for the third formula. More preferably the weight ratio a/b is from 3 to 6 for the first formula, from 7 to 9 for the second formula and from 20 to 30 for the third formula.

The inventors found that a specific combination of non-digestible oligosaccharides consisting of bGOS, lcFOS, 2′-FL, 3-FL, LNT, 3′-SL and 6′-SL as described above improved the microbiota by increasing the amount of bifidobacteria and decreasing the amount of Enterobacteriaceae. Metagenomic analysis indicated that the KEGG orthologs related to carbohydrate metabolism and for enzymes specific for bifidobacteria were also synergistically increased. This again is indicative for a higher percentage of bacteria such genes, i.e. bifidobacteria. Using a combination of 4 HMOs with or without GOS and lcFOS was less effective in increasing bifidobacteria or decreasing Enterobacteriaceae. Therefore, the nutritional composition of the present invention is used for improving the intestinal microbiota in a subject, preferably in an infant or young child, more preferably an infant. Preferably the improving of intestinal microbiota is selected from at least one of increasing bifidobacteria and decreasing pathogenic bacteria, in the intestinal microbiota of a subject, preferably an infant or young child, more preferably an infant, wherein the pathogenic bacteria are preferably Enterobacteriaceae.

The inventors found that the fermentation supernatant of a specific combination of non-digestible oligosaccharides consisting of bGOS, lcFOS, 2′-FL, 3-FL, LNT, 3′-SL and 6′-SL as described above improved the resistance of the intestinal barrier when compared with bGOS plus lcFOS or when compared with the combination of 5 HMOs. An increased resistance of the intestinal barrier is beneficial as it will decrease the translocation of toxins and pathogenic bacteria.

As a result of this improved microbiota and improved intestinal barrier function, the nutritional composition of the present invention will prevent and/or treat gastrointestinal disorders in a subject, preferably an infant or young child, more preferably an infant. Preferably the gastrointestinal disorder is selected from the group consisting of intestinal microbial dysbiosis, in or originating from the intestine and intestinal inflammation.

Thus the present invention also relates to a method for improving the intestinal microbiota in infants or young children, comprising administering to an infant or young child a nutritional composition according to the present invention. This method can be seen as a non-therapeutic method.

For some jurisdictions, this aspect of the invention can be worded as the use of non-digestible oligosaccharides for the manufacture of a nutritional composition according to the present invention, for improving the intestinal microbiota in infants or young children.

Alternatively, this can be worded as a nutritional composition according to the present invention, for use in improving the intestinal microbiota in infants or young children.

In one embodiment, improving the intestinal microbiota is increasing bifidobacteria in the intestine and/or decreasing pathogenic bacteria in the intestine, preferably decreasing Enterobacteriaceae in the intestine. In one embodiment, improving the intestinal microbiota is increasing the intestinal barrier function.

In one aspect, the present invention also relates to a method for increasing the intestinal barrier function in infants or young children, comprising administering to an infant or young child a nutritional composition according to the present invention. This method can be seen as a non-therapeutic method.

For some jurisdictions, this aspect of the invention can be worded as the use of non-digestible oligosaccharides for the manufacture of a nutritional composition according to the present invention, for increasing the intestinal barrier function in infants or young children.

Alternatively, this can be worded as a nutritional composition according to the present invention, for use increasing the intestinal barrier function in infants or young children.

The present invention also relates to a method preventing an/or treating an intestinal disorder, comprising administering to an infant or young child a nutritional composition according to the present invention.

For some jurisdictions, this aspect of the invention can be worded as the use of non-digestible oligosaccharides for the manufacture of a nutritional composition according to the present invention, for preventing an/or treating an intestinal disorder.

Alternatively, this can be worded as a nutritional composition according to the present invention, for use in preventing an/or treating an intestinal disorder.

In one embodiment, the intestinal disorder is selected from the group consisting of microbial dysbiosis, infection in or originating from the intestine and intestinal inflammation.

### DESCRIPTION OF THE FIGURE

FIG. 1: Average relative TEER % (s.d.) of confluent caco-2 monolayers after 48 of treatment with the supernatants of fermented non digestible oligosaccharides standardized to the average relative TEER of bGOS/lcFOS.

### EXAMPLES

### Example 1: Mixtures of a Combination of 5 HMOS and bGOS/lcFOS Synergistically Improve Microbiota of Infants

### Material and Methods

Faecal samples were selected from:
infant 1, a 3 months old breast-fed male infant,
infant 2, a 3 months old breast-fed female infant,
infant 3, a 4.5 months old female infant that was partially breast fed and partially formula fed, and
infant 4, a 4 months old breast-fed female infant

The following mixtures were tested:
component a) bGOS and lcFOS in a weight ratio of 90:10
component b) combination of 5 HMOs: 2′-FL, 3-FL, LNT, 3′-SL and, 6′-SL in a wt ratio of 52, 13, 26, 4 and 5 wt % respectively
mixture according to the invention: bGOS, lcFOS, 2′-FL, 3-FL, LNT, 3′-SL and 6′-SL in a wt ratio of 65.9, 8.7, 13.2, 3.3, 6.6, 1.0 and 1.3 wt % respectively.

For the faecal samples of infants 2 and 3 in addition the following combinations were tested:
combination of 4 HMOs (without LNT): 2′-FL, 3-FL, 3′-SL and 6′-SL in a wt ratio of 39, 23, 10 and 28 wt % respectively
comparative mixture: bGOS, lcFOS, 2′-FL, 3′FL, 3′-SL and 6′-SL in a wt ratio of 64.8 wt % bGOS, 8.6 wt % lcFOS, 10.3% 2′-FL, 6.2% 3-FL, 2.4% 3′-SL and 7.5 wt % 6′-SL respectively.

Colonic Microbiota Medium: Yeast extract 1 g/l, ammoniumsulphate 2 g/l, K2HPO4 2 g/l, NaHCO3 3.2 g/l, NaCl 4.5 g/l, MgSO4.7H2O 0.5 g/l, cysteinHCl 0.5 g/l, CaCl2.2H2O 0.4 g/l, bile salts 25 mg/l, mendione, metal and vitamin solution, haemin (10 mg/l), mucus 2.5 g/l.

Under anaerobic conditions the faecal samples were thawed and a 4% (w/v) suspension of each faecal sample was made in infant adapted Colonic Microbiota medium without carbon source adjusted to breast fed infant stool pH (pH 5.5)) containing 25 mM acetate and 12 mM lactate, 25 mg/L bile acids (Sigma), 2.5 g/L porcine stomach mucin, 15 mmol/L ammonium sulphate, 1 g/l tryptone. The diluted faecal samples were homogenized, allowed to sediment for 5 minutes, then filtered to remove large particles and subsequently filtered over a Millex 100 μm vacuum filter. After initial faecal community stabilization (first 4 hours of fermentation) refeeding/refreshing medium was used without selective acetate and lactate since it will be sufficient produced by microbiota itself.

A 32 well Biolector Pro plate (BOH2 round well, M2P-labs) with pH optodes was used. All fermentation wells of this plate were filled with 1.52 ml of the faecal solutions, one feeding row of the plate was filled with sterile 3M NaOH. The wells of the other feeding row were respectively filled with water (blanc), 10 wt % bGOS:lcFOS 9:1, 10 wt % combination of 4 HMOs, 100/combination of 5 HMOs, 10% bGOS:lcFOS:4 HMOs and bGOS:lcFOS:5 HMOs for continuous night feeding modus. All conditions were performed in fourfold (per column). Plates were sealed with ventilated silicone foil with slits. The plate was incubated (85% moisture, 37° C., 600 rpm, anaerobic) in BioLector Pro. Eighty microliters of 10% (w/v) sterile carbohydrate solutions, as described above, were added, or as a control 80 ul sterile water was added (blanc). The experiment was started with pH at setpoint 5.8 with continuous pH measurement. The pH was controlled at pH 5.4-5.8. After 4 hours the experiment was paused, the fecal slurry of each well was harvested and shortly centrifuged under aseptical anaerobic conditions. The supernatant was frozen for further analyses while the fecal pellet was resuspended in fresh medium with carbohydrates and pipetted back in original well of BOH2 plate. The procedure was repeated again for 3 days, with every day two times manual feeding every 4 hours followed by automatic continuous slow carbohydrate feeding (80 μl overnight) via the microfluidic system. After 3 days the final pellet was harvested and subjected to metagenomics sequencing.

Metagenomic sequencing: Total DNA was extracted from the pellets using the previously described protocol (Codoñer, Ramlrez-Bosca et al. 2018) Scientific Reports 8(1): 3812). A total of 50 ng of DNA was amplified following the 16S Metagenomic Sequencing Library Illumina 15044223 B protocol (Illumina). Nextera XT Library kit (Illumina) was used following the manufacturer's instructions. DNA was simultaneously fragmented and tagged with dual index sequencing adapters. Next, the NovaSeq 6000 sequencing platform was used in a 150 paired end read configuration (NovaSeq Control Software (NCS) version 1.6). Bcl2fastq 2.20 program was used to translate the sequencing reads and removal of sequencing adapters. The Clumpify tool from the BBToolssuite (Bushnell B, BBMap (2015). sourceforge.net/projects/bbmap/) was used to remove optical duplicates and reads with less Phred quality score than Q20 and a length of <50 nucleotides were filtered out using BBMap v38.36 (Bushnell B, 2015). Human genome sequences were filtered using NGLess v1.0.0-Linux64 (Coelho, Alves et al. 2019) Microbiome 7(1): 84) Metaphlan (v.4) (Blanco-Míguez, Beghini et al. 2022 bioRxiv preprint doi: https://doi.org/10.1101/2022.08.22.504593) was used to assign the taxonomy to the reads. The reads from each sample were aligned against single-copy genetic markers present in almost all bacteria. From these alignments, the estimated number of reads contributed by a given clade for each identified taxa were obtained computationally. Per sample the count of reads that were identified as Bifidobacteria and Enterobacteriaceae was divided by the reads that could be assigned to any bacterial taxa to calculate the relative abundance of these (Bifidobacteria and Enterobacteriaceae) bacterial groups.

### Results

Results are shown in tables 1 and 2. Consistently the % of bifidobacteria was increased and the % of Enterobacteriaceae was decreased when the mixture of the 5 HMOs and bGOS and lcFOS was used. This increase in bifidobacteria and decrease of Enterobacteriaceae was higher, and thus synergistic, than expected based on the results of the combination of the 5 HMOs alone or the combination of bGOS plus lcFOS alone. The mixture of bGOS/lcFOS/5 HMOs also outperformed the combination of 4 HMOs and bGOS/lcFOS/4 HMOs mixture. For infants 2 and 3 the total % of bifidobacteria was higher and also a synergistic improvement of bifidobacteria was observed with the mixture of bGOS/lcFOS/5 HMOs. Likewise the % of Enterobacteriaceae was lower with the mixture of bGOS/lcFOS/5 HMOs.

TABLE 1
Bifidobacteria % of total bacteria

| | | 4 HMOs | | | |
|---|---|---|---|---|---|
| | bGOS/ | (minus | | bGOS/lcFOS/ | bGOS/lcFOS/ |
| Infant | lcFOS | LNT) | 5 HMOs | 4 HMOs | 5 HMOs |
| 1 | 71.5 | — | 64.3 | — | 73.7 |
| 2 | 83.7 | 30.5 | 58.7 | 79.8 | 88.5 |
| 3 | 53.2 | 39.8 | 51.3 | 50.8 | 60.4 |
| 4 | 46.6 | — | 70.6 | — | 72.3 |

TABLE 2
Enterobacteriaceae % of total bacteria

| | | 4 HMOs | | | |
|---|---|---|---|---|---|
| | bGOS/ | (minus | | bGOS/lcFOS/ | bGOS/lcFOS/ |
| Infant | lcFOS | LNT) | 5 HMOs | 4 HMOs | 5 HMOs |
| 1 | 16.7 | — | 21.5 | — | 15.9 |
| 2 | 6.8 | 38.1 | 21.2 | 12.6 | 6.0 |
| 3 | 4.4 | 4.6 | 4.2 | 4.1 | 3.4 |
| 4 | 38.7 | — | 21.6 | — | 21.0 |

These results are indicative for an improved and unexpected synergistic effect of the mixture of the specific 5 HMOs combination and bGOS and lcFOS. Bifidobacteria were increased whereas potentially pathogenic bacteria such as the Gram-negative Enterobacteriaceae were decreased. This is indicative of the specificity on improving the microbiota by a mixture consisting of a combination of 5 specific HMOs and bGOS plus lcFOS.

Metagenomic analysis indicated that the KEGG orthologs related to carbohydrate metabolism and for enzymes specific for bifiobacteria were also synergistically increased. This concerned for example (ko:K22397 yjhH, yagE; 2-dehydro-3-deoxy-D-pentonate aldolase [EC:4.1.2.28]), involved in the breakdown of fucose derived from fucosyllactose. This again is indicative for a higher percentage of bacteria such genes, i.e. bifidobacteria.

### Example 2: Contribution of 3-Fucosyllactose in the Combination of 5 HMOs on Bifidogenic Effect and Inhibition of Enterobacteriaceae

### Material and Methods

Faecal samples were selected from:
from infant 1, a 6 months mixed-fed male infant,
from infant 2, a 3 months old breast-fed female infant,

The following mixtures were tested:
combination of 5 HMOs: 2′-FL, 3-FL, LNT, 3′-SL, 6′-SL, in a wt ratio of 52, 13, 26, 4 and 5 wt % respectively
combination of 4 HMOs (without 3-FL): 2′-FL, LNT, 3′-SL, 6′-SL, in a wt ratio of 59.8, 29.9, 4.6 and 5.7 wt % respectively

Samples were subjected to the same protocol and screening for the relative abundance of Bifidobacteria and Enterobacteriaceae bacterial groups as in example 1.

### Results

Selective bifidobacterial plating showed that for both donors the combination of 5 HMOs more CFU bifidobacteria were present compared to the combination of HMOs without 3-FL which confirms that 3-FL contributes to the bifidogenic effect of the combination of 5 HMOs.

Results are further shown in tables 3 and 4.

TABLE 3
Bifidobacteria % of total bacteria

| Infant | 4 HMOs (minus 3-FL) | 5 HMOs |
|---|---|---|
| 1 | 45.4 | 61.7 |
| 2 | 47.9 | 54.8 |

TABLE 4
Enterobacteriaceae % of total bacteria

| Infant | 4 HMOs (minus 3-FL) | 5 HMOs |
|---|---|---|
| 1 | 53.1 | 38.2 |
| 2 | 24.0 | 17.2 |

The % of bifidobacteria was increased and the % of Enterobacteriaceae was decreased for the combination of 5 HMOs compared to the combination of 4 of the HMOs without 3-FL. It can be concluded that 3-FL strongly contributes to the bifidogenic effect and suppression of opportunistic pathogenic bacteria in the family of Enterobacteriaceae.

For the faecal sample of infant 1 also the influence of absence or presence of lcFOS on inhibition of Enterobacteriaceae was tested.

The following combinations were tested:
comparative mixture bGOS/5 HMOS (without lcFOS): bGOS, 2′-FL, 3-FL, LNT, 3′-SL and 6′-SL in a wt ratio of 72.2, 14.4, 3.6, 7.2, 1.1, and 1.4 wt % respectively
mixture according to the invention: bGOS, lcFOS, 2′-FL, 3-FL, LNT, 3′-SL and 6′-SL in a wt ratio of 65.9, 8.7, 13.2, 3.3, 6.6, 1.0 and 1.3 wt % respectively.

TABLE 5
Enterobacteriaceae % of total bacteria

| | bGOS/5 HMOs | bGOS/lcFOS/ |
|---|---|---|
| Infant | (minus lcFOS) | 5 HMOs |
| 1 | 30.7 | 16.4 |

It follows that lcFOS contributes to the suppression of opportunistic pathogenic bacteria in the family of Enterobacteriaceae.

### Example 3: Supernatants of Faecal Fermentation of a Combination of 5 HMOs and GOS/lcFOS Synergistically Improve Intestinal Barrier Function

### Material and Methods

Human colonic carcinoma Caco-2 (ATCC) cells were cultured on transwells (Corning 3460) at a density of 1×10⁵ cells per well. To study the impact on barrier development, cell monolayers were treated 6 days post seeding at confluency, with supernatants extracted from fermented bGOS:lcFOS 9:1, fermented combination of 5 HMOs, and a mixture thereof. The 3 days supernatants of fermentations with faecal slurries from infants 1, 3 and 4 of example 1 were tested (the supernatant of infant 2 was not tested). Prior to addition to the cell monolayers, supernatants were filtered on 2 μm pore size, and diluted 1/10 in DMEM (Gibco) without FCS. The medium was refreshed after 24 h of treatment.

Permeability of the monolayers was assessed after 48 h of treatment by measuring the Trans Epithelial Electrical Resistance (TEER) using the Millicell ERS-2 voltohmmeter (Millipore).

### Results

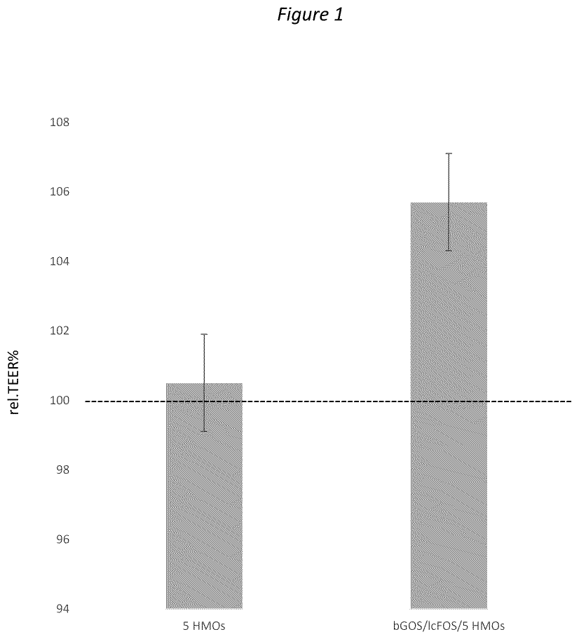

The results are shown in FIG. 1. The values represent the ratio of the TEER value of the treatment arms by their baseline value, followed by standardization to the bGOS/lcFOS arm of the corresponding donor to 100%. Treatments were performed in duplo for each donor, then averaged for the 3 donors.

Surprisingly the TEER value was consistently higher with the supernatants of the fermented specific mixture of the combination of 5 HMOs and bGOS and lcFOS when compared to the combination of 5 HMOs alone or bGOS plus lcFOS alone and a synergistic increase was observed. This effect was observed for the supernatant obtained after fermentation with the microbiota from each individual infant (data not shown).

These results are indicative for an improved and synergistic effect of the specific mixture of a combination of 5 HMOs and bGOS and lcFOS on the gut barrier function after fermentation by the intestinal microbiota.

### Example 4: Infant Formula

Infant formula intended for infants 0-6 month of age, comprising per 100 ml (obtained from reconstituting 13.7 g powder with water):
67 kcal
digestible carbohydrates (mainly lactose): 7.3 g
protein (whey protein, casein): 1.3 g
lipids: 3.4 g
non-digestible oligosaccharides 0.9 g consisting of
   0.7 g GOS/lcFOS in a 7.6:1 wt/wt ratio
   0.2 g of a mixture of HMOs5 consisting of 52 wt % 2′-FL, 13 wt % 3-FL, 26 wt % LNT, 4 wt % 3′-SL and 5 wt % 6′-SL
micronutrients according to directive for infant formulas

### Example 5: Infant Formula

Infant formula intended for infants 0-6 month of age, comprising per 100 ml (obtained from reconstituting 13.7 g powder with water):
66 kcal
digestible carbohydrates (mainly lactose): 7.2 g
protein (whey protein, casein): 1.3 g
lipids: 3.4 g
non-digestible oligosaccharides 0.872 g consisting of
   0.7 g GOS/lcFOS in a 9:1 wt/wt ratio
   0.172 g of a combination of 5 HMOs consisting of 52 wt % 2′-FL, 13 wt % 3-FL, 26 wt % LNT, 4 wt % 3′-SL and 5 wt % 6′-SL
micronutrients according to directive for infant formulas

### Example 6: Follow-on Formula

Follow-on formula intended for infants 6-12 month of age, comprising per 100 ml (obtained from reconstituting 14.4 g powder with water):
68 kcal
digestible carbohydrates (mainly lactose): 8.2 g
protein (whey protein, casein): 1.4 g
lipids: 3.2 g
non-digestible oligosaccharides 0.786 g consisting of
   0.7 g GOS/lcFOS 9:1 wt/wt ratio
   85 mg of a combination of 5 HMOs consisting of 52 wt % 2′-FL, 13 wt % 3-FL, 26 wt % LNT, 4 wt % 3′-SL and 5 wt % 6′-SL
micronutrients according to directive for follow-on formulas

### Example 7: Young Child Formula

Young child formula intended for young children 12 to 36 months of age, comprising per 100 ml (after reconstituting 14.4 g powder with water):
65 kcal
digestible carbohydrates (mainly lactose): 8.3 g
protein (whey protein, casein): 1.3 g
lipids: 2.7 g
non-digestible oligosaccharides 1.243 g consisting of
   1.2 g GOS/lcFOS 9:1 wt/wt ratio
   43 mg of a combination of 5 HMOs consisting of 52 wt % 2′-FL, 13 wt % 3-FL, 26 wt % LNT, 4 wt % 3′-SL and 5 wt % 6′-SL
micronutrients according to directive for follow-on formulas

1. A nutritional composition for infants or young children comprising a mixture of non-digestible oligosaccharides consisting of
component a) beta-galacto-oligosaccharides (bGOS) having an average degree of polymerization (DP) ranging from 3 to 7, and long chain fructo-oligosaccharides (lcFOS) having an average degree of polymerization (DP) ranging from 20 to 40, and
component b) 2′-fucosyllactose (2′-FL), 3-fucosyllactose (3-FL), lacto N tetraose (LNT), 3′ sialyllactose (3′-SL) and 6′ sialyllactose (6′-SL),
wherein component a) consists of 70 to 95 wt % bGOS, based on total weight of component a), and 5 to 30 wt % lcFOS, based on total weight of component a), the sum of bGOS and lcFOS being 100% of the total weight of component a), and
wherein component b) consists of 42 to 62 wt % 2′-FL, 10 to 16 wt % 3-FL, 21 to 31 wt % LNT, 3 to 5 wt % 3′-SL and 4 to 6 wt % 6′-SL, the sum of 2′-FL, 3-FL, LNT, 3′-SL and 6′-SL being 100% of the total weight of component b).

2. The nutritional composition for infants or young children according to claim 1, wherein the composition comprises Bifidobacterium breve.

3. The nutritional composition for infants or young children according to claim 1, comprising 50 to 97.5 wt % component a) based on total weight non-digestible oligosaccharides and 2.5 to 50 wt % component b) based on total weight non-digestible oligosaccharides, the sum of components a) and b) being 100% of the total weight of non-digestible oligosaccharides.

4. The nutritional composition for infants or young children according to claim 1, wherein component a) consists of 85 to 95 wt % bGOS, based on total weight of component a), and 5 to 15 wt % lcFOS, based on total weight of component a), the sum of bGOS and lcFOS being 100% of the total weight of component a).

5. The nutritional composition for infants or young children according to claim 1, wherein the mixture of non-digestible oligosaccharides consists of 60 to 90 wt % GOS, 7.5 to 10 wt % lcFOS, 1.5-15 wt % 2′-FL, 0.4-4 wt % 3-FL, 0.75 to 7.5 wt % LNT, 0.10 to 1.2 wt % 3′-SL and 0.10-1.5 wt % 6′-SL, the sum being 100%.

6. The nutritional composition for infants or young children according to claim 1, comprising, when in ready-to-use form, 120 mg to 2.40 g non digestible oligosaccharides consisting of component a) and b) per 100 ml, or, when in powder form, based on dry weight, 0.86 to 17.14 wt % non digestible oligosaccharides consisting of component a) and b), or, based on energy, 180 mg to 3.60 g non digestible oligosaccharides consisting of component a) and b) per 100 kcal.

7. The nutritional composition for infants or young children according to claim 1, wherein the amount of bGOS plus lcFOS is 100 mg to 2 g per 100 mll; based on dry weight of the composition the amount of bGOS plus lcFOS 0.7 wt % to 14.3 wt %, bGOS plus lcFOS; and/or the amount of bGOS plus lcFOS per 100 kcal is 150 mg to 3 g; and
wherein the amount of the combination of 2′-FL, 3-FL, LNT, 3′-SL and 6′-SL, is 20 to 400 mg per 100 ml; based on dry weight of the composition the amount of the combination of 2′-FL, 3-FL, LNT, 3′-SL and 6′-SL is 0.14 to 2.86 wt %; and/or the amount of the combination of 2′-FL, 3-FL, LNT, 3′-SL and 6′-SL per 100 kcal is 30 to 600 mg.

8. The nutritional composition for infants or young children according to claim 1, comprising 3 to 7 g lipid/100 kcal, 1.25 to 4 g protein/100 kcal and 6 to 20 g digestible carbohydrates/100 kcal.

9. The nutritional composition for infants or young children according to claim 1, which is an infant formula, follow-on formula or young child formula.

10. A method for providing nutrition to an human subject in the first 12 months of life, optionally the first 3 years of life, comprising
a) providing a first nutritional composition for the first 6 months of life, which comprises the mixture of non-digestible oligosaccharides according to claim 1, wherein component a) provides 0.25 to 1.50 g per 100 ml, preferably 0.5 to 1.00 g per 100 ml, and component b) provides 0.03 to 0.40 g per 100 ml, preferably 0.04 t0 0.30 g per 100 ml,
b) providing a second nutritional composition for the age of 6 to 12 months, which comprises the mixture of non-digestible oligosaccharides according to claim 1, wherein component a) provides 0.25 to 1.50 g per 100 ml, and component b) provides 0.03 to 0.30 g per 100 ml, and
c) optionally providing a third nutritional composition for the age of 12 to 36 months, i.e. 1 to 3 years, which comprises the mixture of non-digestible oligosaccharides according to claim 1, wherein component a) provides 0.50 to 2.00 g per 100 ml, and component b) provides 0.02 to 0.30 g per 100 ml, preferably 0.03 to 0.250 g per 100 ml,
with the proviso that the ratio a/b of the second formula is higher than that of the first formula, and the ratio a/b of the optional third formula is higher than that of the second infant formula.

11. The method according to claim 10, wherein the composition comprises Bifidobacterium breve.

12. A method for improving the intestinal microbiota in infants or young children or for increasing the intestinal barrier function or for preventing and/or treating an intestinal disorder in infants or young children comprising the method comprising administering to an infant or young child a nutritional composition according to claim 1.

13. The method according to claim 12, wherein improving the intestinal microbiota is increasing bifidobacteria in the intestine and/or decreasing pathogenic bacteria in the intestine.

14. The method according to claim 12, wherein the intestinal disorder is selected from the group consisting of microbial dysbiosis, infections in or originating from the intestine and intestinal inflammation.
